(19) United States
(12) Patent Application Publication
(10) Pub. No.: US 2018/0193261 A1
(43) Pub. Date: Jul. 12, 2018

(54) METHOD FOR PREPARING HYDROGEL CONTAINING REDUCED GRAPHENE OXIDE

(71) Applicant: Gwangju Institute of Science and Technology, Gwangju (KR)

(72) Inventors: Jae Young LEE, Gwangju (KR); Semin KIM, Jeonju-si (KR); Hyerim JO, Cheonan-si (KR)

(21) Appl. No.: 15/560,739

(22) PCT Filed: Mar. 23, 2016
(86) PCT No.: PCT/KR2016/002931

§ 371 Date: Nov. 29, 2017

(30) Foreign Application Priority Data
Mar. 23, 2015 (KR) 10-2015-0040092

(51) Int. Cl. A61K 9/06; A61K 9/70; A61K 47/36; A61L 27/52

(52) U.S. Cl. CPC A61K 9/06; A61L 27/52; A61K 47/36; A61K 9/70

(57) ABSTRACT

The present invention relates to a method for preparing a natural or synthetic polymer hydrogel loading graphene oxide or graphene, and to the selective and high-capacity adsorption and loading of the hydrogel with respect to a low-molecular weight material or a high-molecular weight material. More specifically, an alginate or polyacrylamide hydrogel loading graphene and a graphene derivative is prepared, wherein the hydrogel can be controlled to enable selective absorption according to the characteristics of an adsorbate by adjusting the reduction degree of graphene oxide, exhibits high adsorption capacity, and is easy to handle as a hydrate. These characteristics can significantly improve the adsorption efficiency with respect to a material in water or an organic phase material, compared with existing hydrogels.

### TECHNICAL FIELD

The present invention relates to a method for preparing a hydrogel including reduced graphene oxide, a hydrogel prepared by the method, and an adsorbent, a drug carrier or a patch for tissue engineering applications including the hydrogel.

### BACKGROUND ART

Technologies associated with the adsorption of materials of various sizes, such as small molecules and large molecules, have been used in a wide range of fields, including removal of water pollutants and drug delivery. Particularly, the development of selective high-capacity adsorbent carriers is considered a key technology in these fields.

Damage caused by water pollutants has long been known for its seriousness. The majority of water pollutants are synthetic organic compounds. Various kinds of such synthetic organic compounds are known, for example, synthetic fertilizers, pesticides, paints, fuels, plastics, and dyes. Synthetic organic compounds are absorbed in vivo and adversely affect human health. Many methods based on chemical precipitation and the use of ion-exchange resins and membranes have been proposed for the removal of organic compounds and heavy metals from river water, underground water, and wastewater. However, these methods suffer from difficulty in treating contaminated water containing large amounts of organic compounds and heavy metals and involve considerable treatment costs. Further, drug delivery materials having hydrophobic functional groups with different characteristics are important factors for effective drug delivery and disease prevention and treatment. Many drug-loading biomaterials have been developed.

As solutions to the problems of conventional treatment technologies, biological treatment methods have emerged for the removal of pollutants. Biosorption of pollutants can be performed by polymeric substances derived from vegetables and animals. Alginic acid is a major component of algal cell walls and has the ability to adsorb heavy metals. Alginic acid chemically belongs to the group of carbohydrates and is a natural polymer having carboxyl groups. Negatively charged alginic acid can adsorb positively charged heavy metals by ion exchange.

In recent years, graphene oxide nanoparticles have been produced economically at a reasonable level. Graphene oxide is widely used for water treatment due to its functional groups, such as hydroxyl and epoxy groups. Particularly, graphene oxide is very effective in adsorbing environmental pollutants, such as heavy metals, cationic organic compounds, and volatile organic compounds. Graphene as a reduction product of graphene oxide is also suitable for the removal of environmental pollutants in the form of aqueous solutions due to its excellent electrical and mechanical properties and large surface area. However, graphene oxide or graphene exists in a suspended state, making it difficult to use as an environmental purification material or drug carrier. There is a possibility that graphene oxide or graphene may cause secondary environmental pollution because of its nanomaterial characteristics. For these reasons, graphene oxide or graphene is rarely used despite its advantageous effects.

### PRIOR ART DOCUMENTS

### Non-Patent Documents

1. Li, J.; Liu, C.-y.; Liu, Y., Au/graphene hydrogel: synthesis, characterization and its use for catalytic reduction of 4-nitrophenol. Journal of Materials Chemistry 2012, 22 (17), 8426-8430.
2. Geng, Z.; Lin, Y.; Yu, X.; Shen, Q.; Ma, L.; Li, Z.; Pan, N.; Wang, X., Highly efficient dye adsorption and removal: a functional hybrid of reduced graphene oxide-Fe3O4 nanoparticles as an easily regenerative adsorbent. Journal of Materials Chemistry 2012, 22 (8), 3527-3535.
3. Fan, J.; Shi, Z.; Lian, M.; Li, H.; Yin, J., Mechanically strong graphene oxide/sodium alginate/polyacrylamide nanocomposite hydrogel with improved dye adsorption capacity. Journal of Materials Chemistry A 2013, 1 (25), 7433-7443.

### DETAILED DESCRIPTION OF THE INVENTION

### Problems to be Solved by the Invention

The present invention has been made in an effort to solve the problems of the prior art and is intended to propose a high-capacity natural or synthetic polymer hydrogel loaded with graphene and a graphene derivative that can selectively adsorb various hydrophilic and hydrophobic small molecules.

### Means for Solving the Problems

One aspect of the present invention provides a method for preparing a reduced graphene oxide (rGO)-containing hydrogel, the method including (A) mixing graphene oxide with a hydrogel precursor and gelling the hydrogel precursor contained in the mixture solution to obtain a graphene oxide-containing hydrogel and (B) reducing the graphene oxide contained in the graphene oxide-containing hydrogel.

A further aspect of the present invention provides a reduced graphene oxide-containing hydrogel including (a) a hydrogel and (b) reduced graphene oxide dispersed in the hydrogel.

Another aspect of the present invention provides an adsorbent including the reduced graphene oxide-containing hydrogel.

Yet another aspect of the present invention provides a myocardial patch including the reduced graphene oxide-containing hydrogel wherein the hydrogel is polyacrylamide.

### Effects of the Invention

The composite of the present invention includes a porous alginic acid or polyacrylamide gel and graphene oxide or reduced graphene oxide with different degrees of reduction loaded in the pores of the porous gel. The composite of the present invention has an outstanding ability to adsorb organic compounds. Due to this ability, the composite of the present invention is used as an adsorbent that can efficiently remove pollutants without causing secondary contamination at a reduced treatment cost. In addition, the composite of the present invention can be used to carry and transport cells, bioactive molecules or other specific substances.

### BRIEF DESCRIPTION OF THE DRAWINGS

FIG. 1 compares a method for preparing a graphene-loaded alginate hydrogel according to the present invention with a conventional method.

FIG. 2 schematically shows a method for preparing a graphene-loaded polyacrylamide hydrogel according to the present invention and the formation of an electrically conductive network of graphene distributed in a hydrogel.

FIG. 3A to 3H shows images of hydrogels prepared using alginic acid in Example 1: especially FIG. 3A shows an alginate hydrogel (Alg), FIG. 3B shows a graphene oxide-loaded alginate hydrogel (GO/Alg), FIG. 3C shows a graphene alginate hydrogel prepared by reduction of GO/Alg for 3 h (r(GO/Alg)₃ₕ), FIG. 3D shows a graphene alginate hydrogel prepared by reduction of GO/Alg for 12 h (r(GO/Alg)₁₂ₕ), FIG. 3E shows a hydrogel prepared by gelation of r(GO/Alg)₃ₕ (rGO₃ₕ/Alg), FIG. 3F shows a hydrogel prepared by gelation of r(GO/Alg)₁₂ₕ (rGO₁₂ₕ/Alg), and FIGS. 3G and 3H are high magnification images of the hydrogels FIGS. 3D and 3F, respectively.

FIG. 4 shows the degrees of reduction of graphene oxide in the order of increasing I_{D}/I_{G} value, which were determined by Raman spectroscopy.

FIG. 5 shows the internal morphologies of graphene-loaded alginate hydrogels prepared in Example 1, which were observed by SEM.

FIG. 6 compares the dye adsorption capacities of hydrogels prepared in Example 1 with those of existing hydrogels.

FIG. 7 shows the adsorption capacities of alginic acid, a graphene oxide-loaded hydrogel, and graphene-loaded hydrogels for various dyes.

FIG. 8 shows images of a polyacrylamide hydrogel, a graphene oxide-loaded polyacrylamide hydrogel, and graphene-loaded polyacrylamide hydrogels prepared by reducing the graphene oxide of the graphene oxide-loaded polyacrylamide hydrogel with vitamin C for 3, 6, 12, and 24 hours in Example 2.

FIG. 9 shows the degrees of reduction of graphene oxide in a graphene oxide-loaded polyacrylamide hydrogel and reduced graphene oxide-loaded polyacrylamide hydrogels prepared by reducing the graphene oxide-loaded polyacrylamide hydrogel with vitamin C for 3, 6, 12, and 24 hours in the order of increasing I_{D}/I_{G} value, which were determined by Raman spectroscopy to analyze the degrees of reduction of the reduced graphene oxide hydrogels.

FIG. 10 shows the internal porous structures and size distributions of a polyacrylamide hydrogel, a graphene oxide-loaded polyacrylamide hydrogel, and reduced graphene oxide-loaded polyacrylamide hydrogels after freeze-drying, which were observed by SEM.

FIG. 11 shows impedance values of distilled water- and PBS-containing polyacrylamide hydrogels, graphene oxide-loaded polyacrylamide hydrogels, and reduced graphene oxide-loaded polyacrylamide hydrogels, which were measured by electrochemistry impedance spectroscopy (EIS). The impedance values of the hydrogels were in the range of about 1 to about 40 kΩ/cm².

FIG. 12 shows Young's moduli of a polyacrylamide hydrogel, a graphene oxide-loaded polyacrylamide hydrogel, and reduced graphene oxide-loaded polyacrylamide hydrogels prepared in Example 2, which were measured using a rheometer. The Young's moduli of the hydrogels were in the range of about 1 to about 30 kPa.

FIG. 13 shows the growth and morphology of myocardial cells (H9c2) cultured in a polyacrylamide hydrogel, a graphene oxide-loaded polyacrylamide hydrogel, and a reduced graphene oxide-loaded polyacrylamide hydrogel for 1 day, fixed, and stained with Phalloidin antibody and DAPI, which were observed using a fluorescence microscope at different magnifications. The reduced graphene oxide-loaded polyacrylamide hydrogel was prepared by the reduction of the graphene oxide-loaded polyacrylamide hydrogel for 24 h. All hydrogels had been washed with DPBS 1× for 2 days before culture.

### BEST MODE FOR CARRYING OUT THE INVENTION

Several aspects and various embodiments of the present invention will now be described in more detail.

One aspect of the present invention is directed to a method for preparing a reduced graphene oxide (rGO)-containing hydrogel, the method including (A) mixing graphene oxide with a hydrogel precursor and gelling the hydrogel precursor contained in the mixture solution to obtain a graphene oxide-containing hydrogel and (B) reducing the graphene oxide contained in the graphene oxide-containing hydrogel.

According to the method of the present invention, a polymer hydrogel including reduced graphene oxide dispersed therein can be prepared without graphene restacking, and as a result, its advantageous effects, such as high adsorption capacity and drug capture capacity, can be maximized.

According to one embodiment, the hydrogel is an alginate hydrogel and the hydrogel precursor is an alginic acid salt. According to an alternative embodiment, the hydrogel is a polyacrylamide hydrogel and the hydrogel precursor is acrylamide.

Examples of such hydrogels include, but are not limited to, alginate hydrogels and polyacrylamide hydrogels. Particularly, an alginate hydrogel is preferred that can advantageously adsorb a dye. A polyacrylamide hydrogel is preferred because it can be advantageously applied to cell and tissue biomaterials, such as myocardial patches.

When it is desired to use an alginate hydrogel, examples of usable hydrogel precursors include, but are not limited to, sodium alginate, calcium alginate, and potassium alginate. Particularly, most preferred is sodium alginate that can be advantageously used in the form of an aqueous solution due to its high solubility.

When it is desired to use a polyacrylamide hydrogel, examples of usable hydrogel precursors include, but are not limited to, acrylamide, vinyl alcohol, and hydroxyethyl methacrylate. Particularly, most preferred is acrylamide that can achieve a wide range of physical elasticity and is effective in mimicking elasticity and physical properties comparable to those of cardiac muscle.

The degree of cross-linking of the hydrogel can be controlled by varying the gelation conditions, and as a result, the physical properties (for example, adsorption capacity and drug capture capacity) of the hydrogel can be maximized or elaborately modified.

According to another embodiment, the hydrogel is an alginate hydrogel and the cross-linking agent is calcium chloride. When the hydrogel is an alginate hydrogel, examples of polyvalent cations suitable for gelation include, but are not limited to, calcium, barium, magnesium, iron, and strontium ions. Particularly, most preferred is calcium chloride that is advantageous for rapid gelation.

According to another embodiment, the hydrogel is a polyacrylamide hydrogel and the cross-linking agent is ammonium peroxosulfate. When the hydrogel is a polyacrylamide hydrogel, examples of polyvalent cationic compounds suitable for gelation include, but are not limited to, ammonium peroxosulfate, potassium peroxosulfate, sodium peroxide, and riboflavin. Particularly, most preferred is ammonium peroxosulfate that immediately reacts as soon as it is dissolved in water, thus being effective in achieving excellent physical properties of the hydrogel.

According to another embodiment, the graphene oxide-containing hydrogel is reduced by immersion in a reducing solution. Alternatively, the reduction may be performed by bringing the graphene oxide-containing hydrogel into contact with HI gas or irradiation the graphene oxide-containing hydrogel with near-infrared light. However, there is no restriction on the method for reducing the graphene oxide-containing hydrogel. It is preferred to reduce the graphene oxide-containing hydrogel by immersion in a reducing solution because the hydrogel includes water.

According to another embodiment, the reducing solution includes L-ascorbic acid. The reducing solution includes a reducing agent and examples of usable reducing agents include, but are not limited to, L-ascorbic acid (or vitamin C) and hydrazine. Particularly, the use of non-toxic L-ascorbic acid is most preferred.

The ratio of graphene oxide to reduced graphene oxide (GO:rGO) in the reduced graphene oxide-containing hydrogel can be controlled by varying the degree of reduction of graphene oxide. This enables control over the overall hydrophobicity of the reduced graphene oxide-containing hydrogel so that the physical properties (for example, adsorption capacity and drug capture capacity) of the reduced graphene oxide-containing hydrogel can be maximized or elaborately modified.

According to another embodiment, the hydrogel is an alginate hydrogel and the reduction is performed such that the ratio (I_{D}/I_{G}) of the intensity of D band (I_{D}) to the intensity of G band (I_{G}) of the reduced graphene oxide-containing hydrogel is from 1.6 to 2.2, as determined by Raman spectroscopy, and the C/O elemental ratio of the reduced graphene oxide-containing hydrogel is from 1.6 to 1.9, as determined by XPS.

When the hydrogel is an alginate hydrogel, the reduction is performed such that the ratio (I_{D}/I_{G}) of the intensity of D band (I_{D}) to the intensity of G band (I_{G}) of the reduced graphene oxide-containing hydrogel is from 1.6 to 2.2, as determined by Raman spectroscopy, and the C/O elemental ratio of the reduced graphene oxide-containing hydrogel is from 1.6 to 1.9, as determined by XPS. This appropriate reduction was confirmed to be advantageous because the adsorption capacity of the reduced graphene oxide-containing hydrogel for large molecules as well as small molecules can be maximized.

Particularly, it is preferable to perform the reduction such that the I_{D}/I_{G} ratio is in the range of 1.7 to 2.1 and the C/O elemental ratio is in the range of 1.65 to 1.75. It is most preferable to perform the reduction such that the I_{D}/I_{G} ratio is in the range of 1.8 to 2.1 and the C/O elemental ratio is in the range of 1.7 to 1.75. Particularly, it was confirmed that when the I_{D}/I_{G} ratio and the C/O elemental ratio are within the respective most preferable ranges defined above, the hydrophobicity of the reduced graphene oxide-containing hydrogel is enhanced, which maximize the adsorption capacity and drug delivery capacity of the reduced graphene oxide-containing hydrogel, and the multilayer internal structure of the hydrogel is maintained in a state in which the reduced graphene oxide is uniformly dispersed in the hydrogel, which allows the adsorption capacity and drug delivery capacity of the reduced graphene oxide-containing hydrogel to remain substantially unchanged despite repeated cycles of adsorption/desorption or drug capture/release cycle with time, unlike when the I_{D}/I_{G} ratio and the C/O elemental ratio are outside the respective most preferable ranges.

According to another embodiment, the hydrogel is a polyacrylamide hydrogel and the reduction is performed such that the ratio (I_{D}/I_{G}) of the intensity of D band (I_{D}) to the intensity of G band (I_{G}) of the reduced graphene oxide-containing hydrogel is from 0.95 to 1.5, as determined by Raman spectroscopy.

When the hydrogel is a polyacrylamide hydrogel, it is advantageous to perform the reduction such that the I_{D}/I_{G} ratio of the reduced graphene oxide-containing hydrogel is from 0.95 to 1.5, as determined by Raman spectroscopy.

Particularly, the reduction is performed such that the I_{D}/I_{G} ratio is preferably in the range of 1 to 1.4 and more preferably in the range of 1.1 to 1.35. Particularly, it was confirmed that when the I_{D}/I_{G} ratio and the C/O elemental ratio are within the respective most preferable ranges defined above, the reduction of graphene oxide to graphene contributes to an increase in electrical conductivity, unlike when the I_{D}/I_{G} ratio and the C/O elemental ratio are outside the respective most preferable ranges.

A further aspect of the present invention is directed to a reduced graphene oxide-containing hydrogel including (a) a hydrogel and (b) reduced graphene oxide dispersed in the hydrogel.

According to one embodiment, the reduced graphene oxide-containing hydrogel is prepared by reducing graphene oxide contained in the hydrogel (a).

According to a further embodiment, the reduced graphene oxide-containing hydrogel is prepared by the method.

Another aspect of the present invention is directed to an adsorbent including the reduced graphene oxide-containing hydrogel.

Preferably, the adsorbent of the present invention targets a hydrophobic organic compound or a zwitterionic organic compound. Examples of such hydrophobic organic compounds include, but are not limited to, RB and R110. Examples of such zwitterionic organic compounds include, but are not limited to, R6G and R123.

Another aspect of the present invention is directed to a drug carrier including the reduced graphene oxide-containing hydrogel.

Yet another aspect of the present invention is directed to a myocardial patch including the reduced graphene oxide-containing hydrogel. When the hydrogel is polyacrylamide, the reduced graphene oxide-containing hydrogel can be applied to biomaterials for tissue engineering applications. The application of the reduced graphene oxide-containing hydrogel is not limited to myocardial patches.

### MODE FOR CARRYING OUT THE INVENTION

The present invention will be explained in more detail with reference to the following examples. However, these examples are not to be construed as limiting or restricting the scope and disclosure of the invention. It is to be understood that based on the teachings of the present invention including the following examples, those skilled in the art can readily practice other embodiments of the present invention whose experimental results are not explicitly presented. It will also be understood that such modifications and variations are intended to come within the scope of the appended claims.

### EXAMPLES

### Example 1: Preparation and Reduction of Graphene-Loaded Alginate Hydrogel

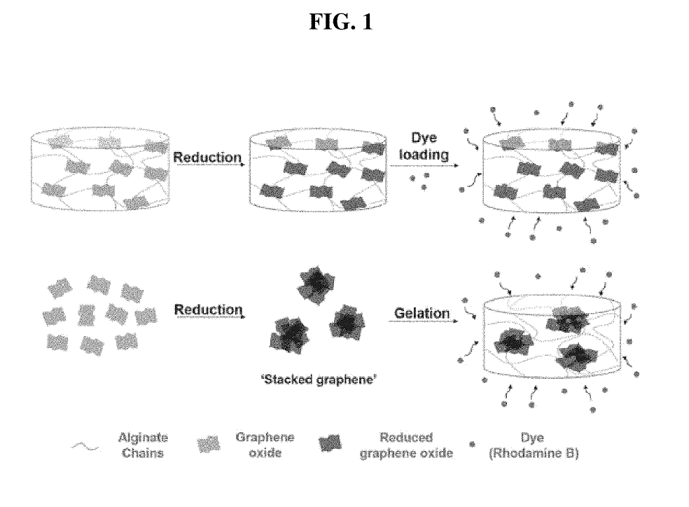

In this example, a graphene-loaded alginate hydrogel was prepared by mixing graphene oxide with an aqueous alginic acid solution to obtain a graphene oxide-containing hydrogel and reducing the graphene oxide. The graphene-loaded alginate hydrogel was effective in terms of adsorption capacity over a reduced graphene oxide-loaded hydrogel prepared by a conventional method. Since reduced graphene oxide tends to stack due to its hydrophobic interaction, effective dye adsorption capacity of conventional reduced graphene oxide-loaded hydrogels cannot be expected despite the presence of the same amount of graphene oxide (see FIG. 1).

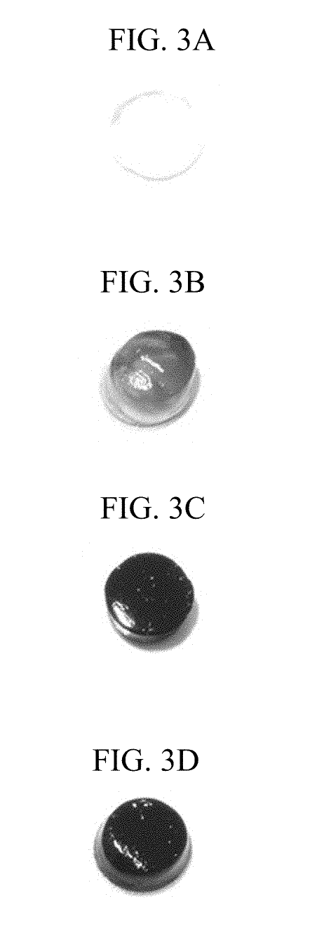

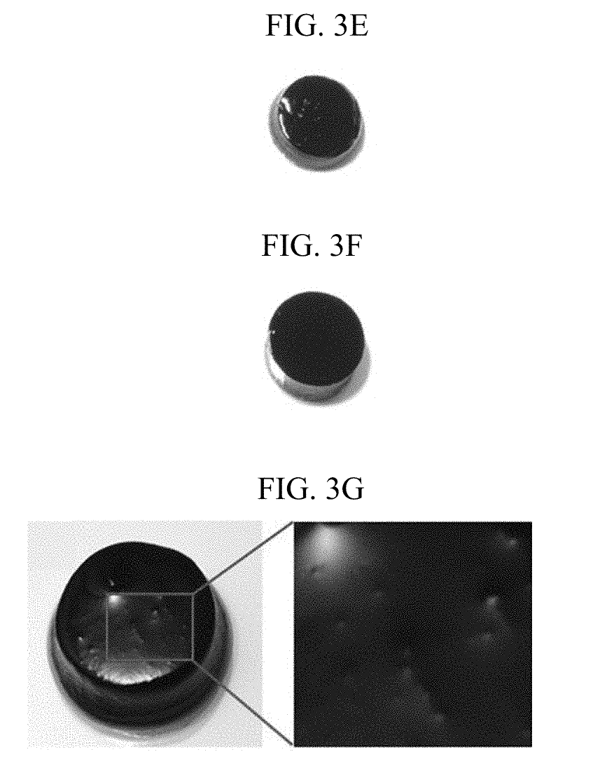

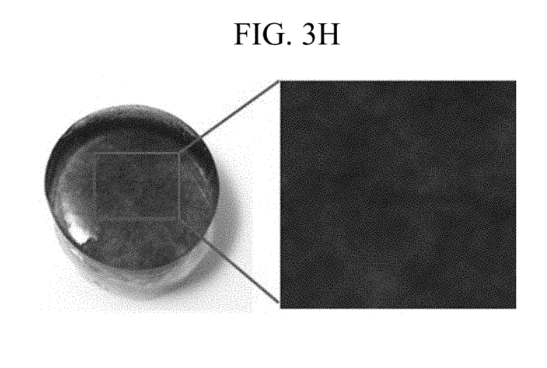

Specifically, sodium alginate (FMC biopolymer) was added in such an amount that its concentration in a 2 mg/mL aqueous solution of graphene oxide (Graphene supermarket) was 2 wt %. The mixture solution was stirred at 200 rpm for 12 h. Thereafter, the alginic acid/graphene oxide mixture solution was immersed in 10 mL of a 0.03 M calcium chloride (CaCl₂) solution as a polyvalent cationic solution for 12 h to prepare an alginate hydrogel. The hydrogel was washed twice with distilled water. The hydrogel was perforated with a 0.6-cm diameter punch to obtain a gel sample of the same size. The gel sample was immersed in a 2 mg/mL aqueous solution of vitamin C at 37° C. for 1 h to prepare a reduced graphene oxide-containing hydrogel. Another reduced graphene oxide-containing hydrogel was prepared in the same manner as described above. except that the immersion time was changed to 12 h. The vitamin C was removed from the reduced graphene oxide-loaded alginate hydrogels using distilled water (see FIGS. 3A to 3H).

FIGS. 3A to 3H show images of the hydrogels prepared using alginic acid: (a) the alginate hydrogel (Alg), (b) the graphene oxide-loaded alginate hydrogel (GO/Alg), (c) the graphene alginate hydrogel prepared by reduction of GO/Alg for 3 h (r(GO/Alg)₃ₕ), (d) the graphene alginate hydrogel prepared by reduction of GO/Alg for 12 h (r(GO/Alg)₁₂ₕ), (e) the hydrogel prepared by gelation of r(GO/Alg)₃ₕ (rGO₃ₕ/Alg), (f) the hydrogel prepared by gelation of r(GO/Alg)₁₂ₕ (rGO₁₂ₕ/Alg), and (g) and (h) are high magnification images of the hydrogels (d) and (f), respectively.

When the graphene oxide was reduced with vitamin C, the gels turned from brown to black. FIG. 3G shows the black hydrogel in which the graphene oxide was uniformly distributed without stacking. In contrast, FIG. 3H shows visible stacking of the graphene oxide when reduced.

### Test Example 1-1: Measurement of Degrees of Reduction of the Graphene-Loaded Alginate Hydrogels

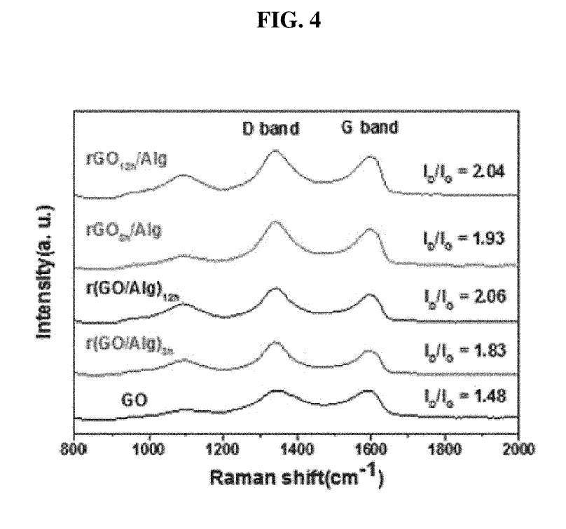

Each of the graphene-loaded alginate hydrogels was dissolved in a 0.1 M EDTA solution. Thereafter, the solution was centrifuged at 8,000 rpm for 10 min and residual EDTA was removed using distilled water. The aqueous graphene solution was dropped onto slide glass, dried, and measured for the degree of reduction of graphene oxide by Raman spectroscopy (a UniThink Inc., UniRaman, 514 nm laser) (see FIG. 4).

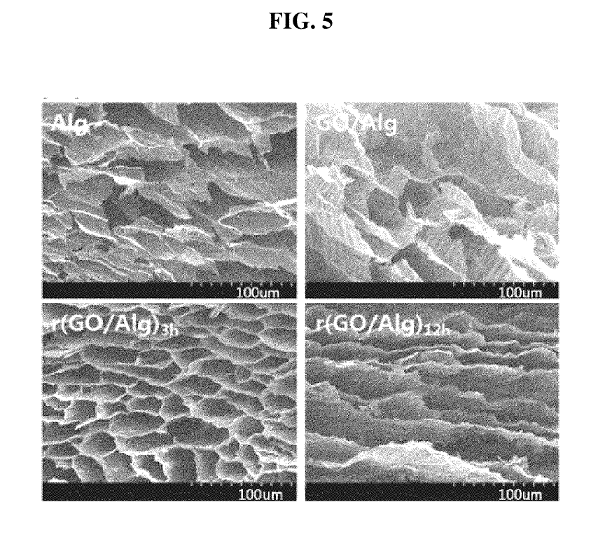

Since the functional groups of the alginate hydrogel varies depending on the degree of reduction of graphene oxide, Raman spectroscopy was used to measure the degrees of reduction of graphene loaded in the alginate hydrogel in order to investigate the adsorption capacity of the alginate hydrogel. D band (1350 cm⁻¹) and G band (1590 cm⁻¹) can be observed in the Raman spectra. The degree of reduction of graphene can be determined by an increase in the ratio (I_{D}/I_{G}) of the intensity of D band to the intensity of G band. Referring to FIG. 4, the I_{D}/I_{G} value increased gradually in the order of GO/Alg (1.48), r(GO/Alg)₃ₕ (1.83) and r(GO/Alg)₁₂ₕ (2.06). The reduced graphene oxide-containing hydrogels rGO₃ₕ/Alg and rGO₁₂ₕ/Alg had I_{D}/I_{G} values of 1.93 and 2.04, respectively. That is, a longer reduction time leads to an increase in I_{D}/I_{G} value, indicating an increase in the degree of reduction (see FIG. 5).

XPS analysis also revealed the reduction of the alginate hydrogels. The C/O elemental ratio of GO/Alg was 1.49 and that of r(GO/Alg)₁₂ₕ was higher (1.73), indicating more reduction of the graphene oxide in r(GO/Alg)₁₂ₕ.

### Test Example 1-2: Analysis of Internal Morphologies of the Graphene-Loaded Alginate Hydrogels

The graphene-loaded alginate hydrogels were cooled with liquid nitrogen and freeze-dried for 3 days. The dry hydrogels were treated with platinum and analyzed by scanning electron microscopy (SEM) (see FIG. 5).

The SEM images of FIG. 5 show the internal structures of the hydrogels. The internal structures of the Alg, GO/Alg, r(GO/Alg)₃ₕ, and r(GO/Alg)₁₂ₕ hydrogel samples reveal that as the reduction of graphene oxide proceeded, graphene oxide layers were stacked due to the hydrophobic interaction of graphene oxide. No stacking of the reduced graphene was observed.

### Test Example 1-3: Measurement of Dye Adsorption Capacities of the Graphene-Loaded Alginate Hydrogels

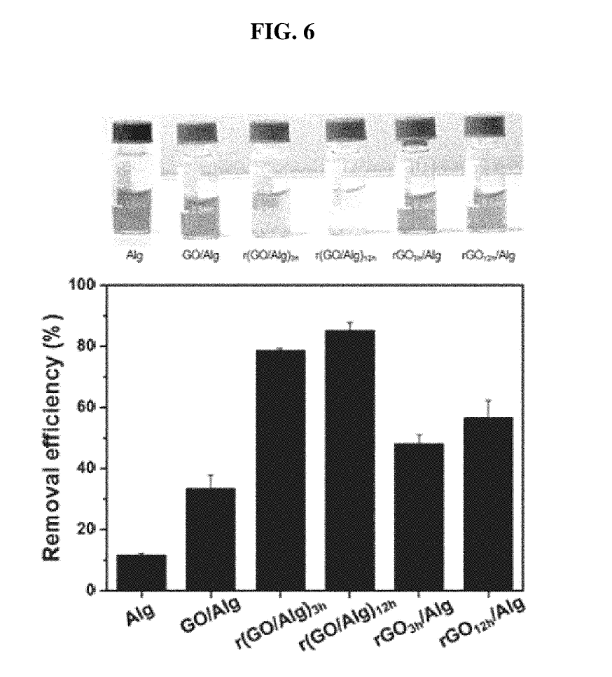

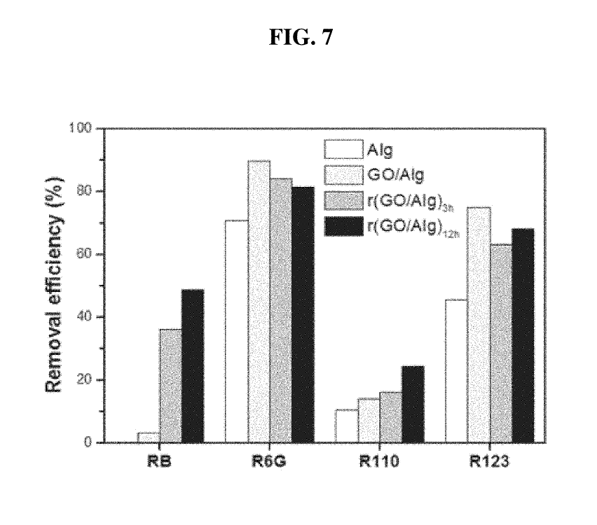

Rhodamine dyes (Sigma-Aldrich) at various concentrations (20-400 mg/L) and a fixed pH of 6.5 were prepared. The hydrogels (each 6-mm diameter) were immersed in aqueous dye solutions at room temperature for 3 days and their dye adsorption capacities were investigated. Standard curves were plotted for Rhodamine B, Rhodamine 6G, Rhodamine 110, and Rhodamine 123 at maximum wavelengths of 540, 530, 500, and 500 nm, respectively, to determine the amounts of the dyes adsorbed (see FIGS. 6 and 7).

This test was conducted to investigate how much the adsorption capacities of the hydrogels for the rhodamine dyes varied depending on the degree of reduction. The test results show that as the reduction proceeded, the removal efficiency of the dye increased. The efficiencies of the reduced graphene-loaded hydrogels were confirmed to be lower than those of the hydrogels prepared by reduction of the graphene oxide-containing hydrogel. The more effective dye adsorption capacities of the reduced hydrogels appear to be because the hydrophobic rhodamine dyes were hydrophobically bound to the hydrophobic reduced graphene oxide (see FIG. 6).

The adsorption capacities of the graphene-loaded hydrogels were tested using rhodamine dyes with different functional groups. Rhodamine B (RB), Rhodamine 110 (R110), Rhodamine 6G (R6G), and Rhodamine 123 (R123) having different functional groups interacted differently with the graphene-loaded hydrogels. The greater hydrophobic interactions of zwitterionic dyes R6G and R123 explain their outstanding dye adsorption capacities. This appears to be because the number of sites of the reduced graphene oxide capable of hydrogen bonding or ionic bonding decreased as the reduction proceeded, resulting in an increase in the adsorption capacities of the graphene-loaded hydrogels for the hydrophobic dyes (see FIG. 7).

### Example 2: Preparation and Reduction of Graphene-Loaded Polyacrylamide Hydrogel

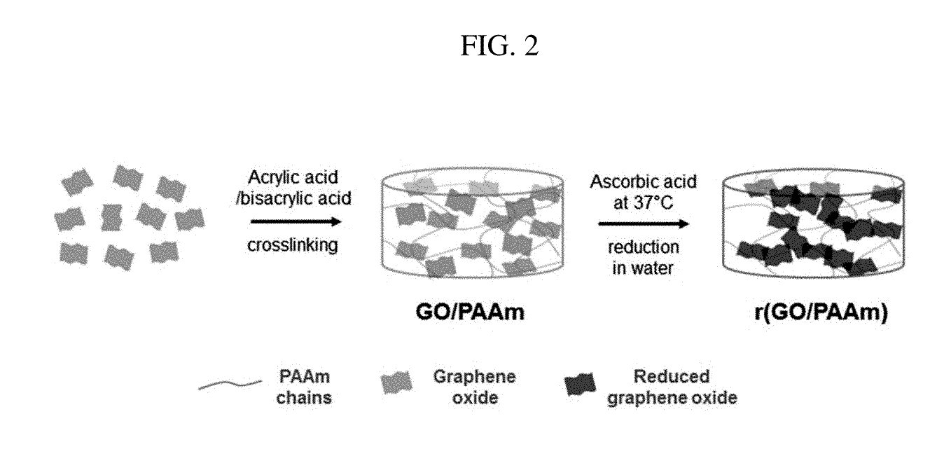

In this example, a graphene-loaded polyacrylamide hydrogel was prepared by mixing graphene oxide with an aqueous acrylamide solution to obtain a graphene oxide-containing hydrogel and reducing the graphene oxide with vitamin C. The graphene-loaded polyacrylamide hydrogel can be applied to myocardial patches with uniform electrical conductivity and elasticity, unlike a reduced graphene oxide-loaded hydrogel prepared by a conventional method (see FIG. 2).

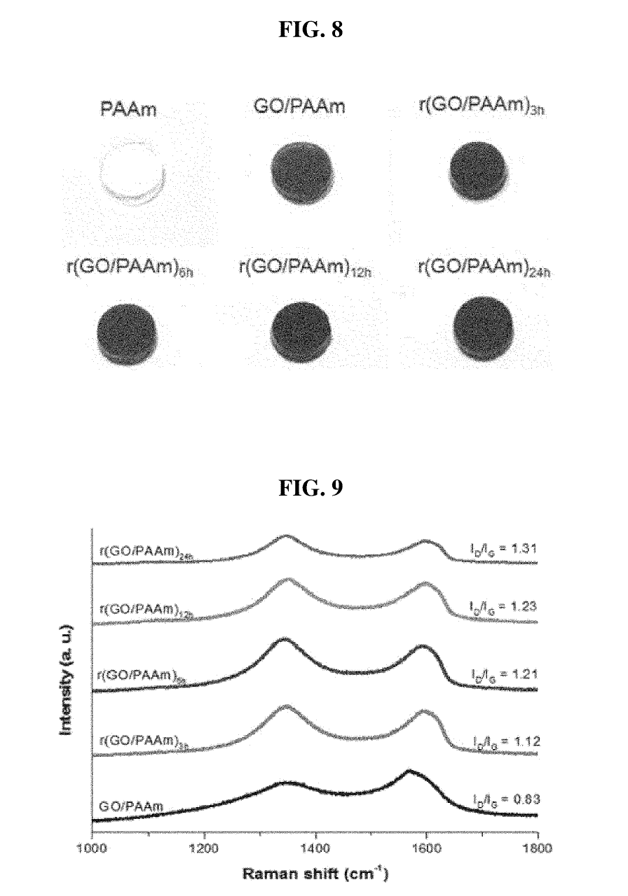

Specifically, 2.64 mL of an aqueous solution containing acrylamide and bisacrylamide in a weight ratio of 29:1, 4 mL of a 6 mg/mL graphene oxide solution (Graphene supermarket), and 1.36 mL of distilled water were mixed at 0° C., and 80 μL of ammonium peroxosulfate was added thereto. Thereafter, 7-8 mL of the resulting solution was placed in casting stands (Bio-rad) at 1-mm intervals and gelled at 60° C. for 4 h. The resulting hydrogels were reduced with vitamin C at 37° C. for different times (3, 6, 12, and 24 h) (see FIG. 8).

FIG. 8 shows the hydrogel samples prepared using acrylamide. When reduced with vitamin C, the hydrogels turned from brown to black with increasing degree of reduction (see FIG. 8).

### Test Example 2-1: Analysis of Internal Morphologies of the Graphene-Loaded Polyacrylamide Hydrogels

The graphene-loaded polyacrylamide hydrogels were rapidly cooled with liquid nitrogen and freeze-dried. The dry hydrogels were finely ground using a mortar and pestle and mixed with distilled water. A small amount of the solution including each of the finely ground hydrogels was dropped onto slide glass, the distilled water was evaporated on an electric heater, and the degree of reduction of the hydrogel was determined by Raman spectroscopy (a UniThink Inc., UniRaman, 514 nm laser) (see FIG. 9).

The electrical conductivity increases with increasing degree of reduction of graphene oxide. Thus, the degrees of reduction of the graphene oxide-loaded polyacrylamide hydrogels were determined by Raman spectroscopy and the impedance values of the hydrogels were measured by electrochemistry impedance spectroscopy. As a result, it was confirmed that the electrical conductivity increased with increasing reduction time. Referring to FIG. 9, the I_{D}/I_{G} value increased in the order of GO/PAAm (0.83), r(GO/PAAm)₃ₕ (1.12), r(GO/PAAm)₆ₕ (1.21), r(GO/PAAm)₁₂ₕ (1.23), and r(GO/PAAm)₂₄ₕ (1.31). That is, as the reduction time increased, the I_{D}/I_{G} value increased, indicating more reduction of graphene oxide.

### Test Example 2-2: Measurement of Degrees of Reduction of the Graphene-Loaded Polyacrylamide Hydrogels

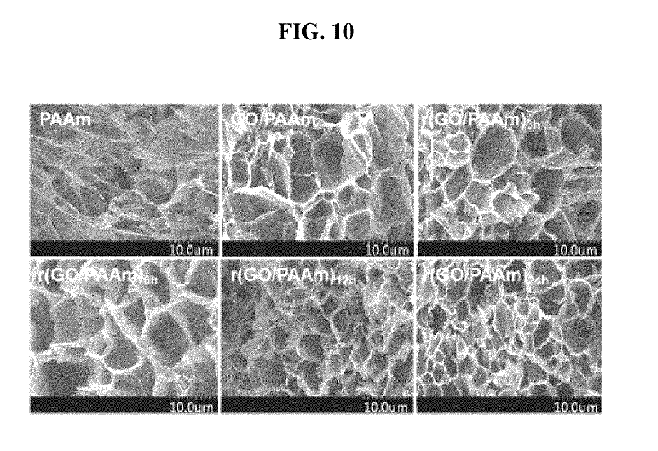

The graphene-loaded polyacrylamide hydrogels were rapidly cooled with liquid nitrogen and freeze-dried. The dry hydrogels were treated with platinum and their internal structures were analyzed by scanning electron microscopy (SEM) (see FIG. 10).

FIG. 10 shows the internal structures of the hydrogels, which were observed by SEM. The three-dimensional highly porous internal structures of the hydrogels reveal that the hydrogels mimic the internal structure (for example, large surface area) of living tissue and are thus suitable for use as biomaterials.

### Test Example 2-3: Measurement of Physical Properties of the Graphene-Loaded Polyacrylamide Hydrogels

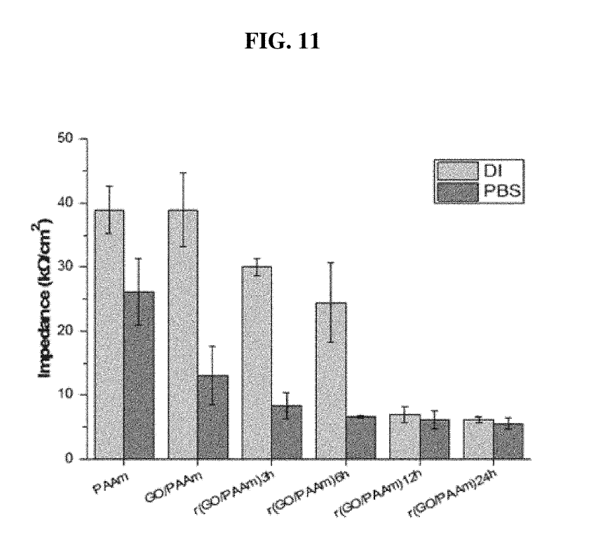

Each of the polyacrylamide hydrogel, the graphene oxide-loaded polyacrylamide hydrogel, and the graphene oxide-loaded polyacrylamide hydrogels prepared by reduction for different times was designed to have a diameter of 12 mm After water removal, the Young's modulus of the hydrogel was measured using a rheometer by sweeping frequencies over the range of 10-0.1 Hz. At this time, the between the hydrogel and the load cell was set to 0.8 mm (see FIG. 11).

Referring to FIG. 11, the hydrogels were allowed to swell in distilled water and PBS and their impedance values were measured. As a result, the electrical conductivity increased with increasing reduction time (i.e. with increasing degree of reduction). When swollen in distilled water, the hydrogels were reduced and became electrically conductive. In addition, the measured electrical conductivities of the hydrogels swollen in PBS show that the hydrogels were electrically conductive even in the environment similar to body fluid.

### Test Example 2-4: Measurement of Electrical Conductivities of the Graphene-Loaded Polyacrylamide Hydrogels

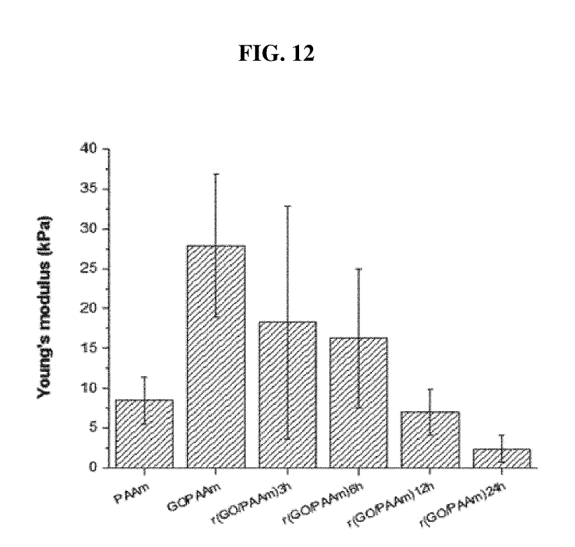

Each of the polyacrylamide hydrogel, the graphene oxide-loaded polyacrylamide hydrogel, and the graphene oxide-loaded polyacrylamide hydrogels prepared by reduction for different times was designed to have a diameter of 8 mm. The hydrogel was interposed between two ITO glass slides, each of which was attached with a copper tape and connected to an electrode. The distance between the two ITO glass slides was set to 0.45 mm, the slides were pressed with an object weighing ≥200 g such that the entire area of the hydrogel was in contact with the ITO glass, and the impedance of the hydrogel was measured at 1 Hz by electrochemistry impedance spectroscopy (EIS) while applying an alternating current to the hydrogel (see FIG. 12).

For use of a hydrogel as an electrically conductive and elastic myocardial patch, the hydrogel is required to possess a modulus similar to that of cardiac muscle. The moduli of the graphene oxide-loaded polyacrylamide hydrogels were measured to evaluate the physical properties of the hydrogels. As a result, the hydrogels had Young's moduli of ˜1-30 kPa, which are similar to that (˜1-100 kPa) of cardiac muscle. These results lead to the conclusion that the graphene oxide-loaded polyacrylamide hydrogels can be utilized as myocardial patches (see FIG. 12).

### Test Example 2-5: Culture of Myocardial Cells in the Graphene-Loaded Polyacrylamide Hydrogels

Each of the polyacrylamide hydrogel, the graphene oxide-loaded polyacrylamide hydrogel, and the graphene oxide-loaded polyacrylamide hydrogel prepared by reduction for 24 h was designed to have a diameter of 8 mm. The hydrogel was placed on a 48-well cell culture dish, disinfected and sterilized with ethanol and UV light, and washed using Dulbecco's Phosphate-Buffered Saline 1× (DPBS, Gibco) for 2 days. The surface of the hydrogel was dried in an incubator for 5-10 min Myocardial cells (H9c2) were mixed in ≤10 μL of a cell culture solution, lightly dropped onto the hydrogel, and cultured in an incubator for 3-5 min. Thereafter, ˜300-400 μL of a cell culture solution was added. Cells were cultured for 1 day. After completion of the culture, cells was fixed in glutaraldehyde 4% (Sigma-aldrich) solution and stained with Phalloidin antibody and 4,6-diamidino-2-phenylindole (DAPI, Sigma-aldrich). The morphology and F-actin of the myocardial cells cultured in the hydrogel were observed.

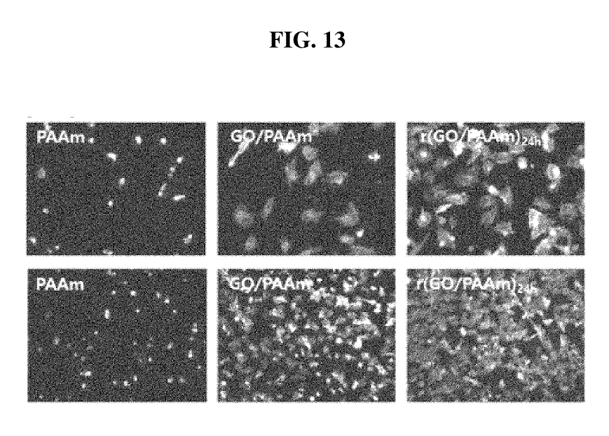

To evaluate the biocompatibility of the graphene oxide-loaded polyacrylamide hydrogel and the applicability of the hydrogel to a myocardial patch, H9c2 and cardiac myoblasts were cultured for 24 h. It was confirmed that H9c2 cells were well adherent to the hydrogel even without the application of extracellular matrix and the hydrogel contributed to cell growth. Particularly, H9c2 cells were most adherent to the reduced graphene oxide-loaded polyacrylamide hydrogel prepared by reduction of graphene oxide, indicating that the electrical conductivity of the hydrogel contributes to intercellular interaction. Therefore, these results concluded that the hydrogel is effective in cell adhesion and cell growth (see FIG. 13).

### Test Example 2-6: Measurement of the Dye Adsorption Capacities of the Graphene-Loaded Polyacrylamide Hydrogels

Rhodamine dyes at various concentrations (50 mg/L) were prepared. The hydrogels (each 6-mm diameter) were immersed in aqueous dye solutions at room temperature for 3 days and their dye adsorption capacities were investigated. Standard curves were plotted for Rhodamine B, Rhodamine 6G, and Rhodamine 123 at maximum wavelengths of 540, 530, and 500 nm, respectively, to determine the amounts of the dyes adsorbed.

1. A method for preparing a reduced graphene oxide (rGO)-containing hydrogel, the method comprising (A) mixing graphene oxide with a hydrogel precursor and gelling the hydrogel precursor contained in the mixture solution to obtain a graphene oxide-containing hydrogel and (B) reducing the graphene oxide contained in the graphene oxide-containing hydrogel.

2. The method according to claim 1, wherein the hydrogel is an alginate hydrogel and the hydrogel precursor is an alginic acid salt.

3. The method according to claim 1, wherein the hydrogel is a polyacrylamide hydrogel and the hydrogel precursor is acrylamide.

4. The method according to claim 3, wherein the gelation is performed by adding a cross-linking agent to the mixture solution.

5. The method according to claim 4, wherein the hydrogel is an alginate hydrogel and the cross-linking agent is calcium chloride.

6. The method according to claim 5, wherein the hydrogel is a polyacrylamide hydrogel and the cross-linking agent is ammonium peroxosulfate.

7. The method according to claim 4, wherein the graphene oxide-containing hydrogel is reduced by immersion in a reducing solution.

8. The method according to claim 7, wherein the reducing solution comprises L-ascorbic acid.

9. The method according to claim 7, wherein the hydrogel is an alginate hydrogel and the reduction is performed such that the ratio (I_{D}/I_{G}) of the intensity of D band (I_{D}) to the intensity of G band (I_{G}) of the reduced graphene oxide-containing hydrogel is from 1.6 to 2.2, as determined by Raman spectroscopy, and the C/O elemental ratio of the reduced graphene oxide-containing hydrogel is from 1.6 to 1.9, as determined by XPS.

10. The method according to claim 7, wherein the hydrogel is a polyacrylamide hydrogel and the reduction is performed such that the ratio (I_{D}/I_{G}) of the intensity of D band (I_{D}) to the intensity of G band (I_{G}) of the reduced graphene oxide-containing hydrogel is from 0.95 to 1.5, as determined by Raman spectroscopy.

11. A reduced graphene oxide-containing hydrogel comprising (a) a hydrogel and (b) reduced graphene oxide dispersed in the hydrogel.

12. The reduced graphene oxide-containing hydrogel according to claim 11, wherein the reduced graphene oxide-containing hydrogel is prepared by reducing graphene oxide contained in the hydrogel (a).

13. An adsorbent comprising the reduced graphene oxide-containing hydrogel according to claim 12.

14. A drug carrier comprising the reduced graphene oxide-containing hydrogel according to claim 12.

15. A myocardial patch comprising the reduced graphene oxide-containing hydrogel according to claim 12 wherein the hydrogel is polyacrylamide.
